(19) United States
(12) Patent Application Publication
(10) Pub. No.: US 2008/0039677 A1
(43) Pub. Date: Feb. 14, 2008

(54) Portable Health Device and Associated Methods

(76) Inventor: Arthur Henry Adams, Marina Del Ray, CA (US)

Correspondence Address: THE TORPY GROUP, P.L., 202 N. HARBOR CITY BLVD., SUITE 200, MELBOURNE, FL, 32935, US

(21) Appl. No.: 11/740,605

(22) Filed: Apr. 26, 2007

Related U.S. Application Data
(60) Provisional application No. 60/795,224, filed on Apr. 26, 2006.

(51) Int. Cl. A61N 2/02

(52) U.S. Cl. 600/13; 600/15

(57) ABSTRACT

A portable health device comprises a housing at least one power source connected to the housing, a frequency oscillation device in communication with the power source, and at least one electromagnet carried by the housing and in communication with the at least one power source. The frequency oscillation device may be adapted to convert power from the power source to a pulse having a predetermined frequency. The pulse may charge the at least one electromagnet to emit a pulsed electromagnetic wave onto a user.

### RELATED APPLICATIONS

This application claims the benefit of U.S. Provisional Patent Application Ser. No. 60/795,224 filed on Apr. 26, 2007, the entire contents of which are incorporated herein by reference.

### FIELD OF THE INVENTION

The present invention relates to portable electronic health devices that emit electro magnetic waves and, more particularly, to jewelry that emits electro magnetic waves onto a wearer's body to optimize brain waves.

### BACKGROUND OF THE INVENTION

There are four main brainwave frequencies in the human brain. Alpha, Beta, Delta, and Theta. Delta and Theta waves are related to sleep states, Beta waves are related to ordinary consciousness, and Alpha waves are present after meditation, before peak sports experiences, and are present at times where tasks may be accomplished without effort. The main frequency for Alpha brain waves is approximately 7.8 HZ.

There have been many approaches with sound and light to try to affect brain wave states. Alpha waves are particularly interesting because of a number of studies that have shown that the earth vibrates at an alpha frequency (the Schumann resonance). Further, alpha frequency has been shown to be present in peak sports moments, meditation and in instances of measured radiation emitting from healers hands. It has long been known that brain waves can be affected by other vibrations. There exists a natural process known as brain entrainment and characterized by synchronization of the brain with nearby vibrations that are closest to its frequencies.

There exist devices made to affect brain waves. For example, U.S. Pat. No. 5,409,445 to Rubins discloses a brain wave synchronizer, The brain wave synchronizer uses pulsed lights seen through special eyeshades and sounds played through stereo earphones to synchronize a user's brainwaves. The brain wave synchronizer is connected to either a computer or a CD player. This particular device, however, may be expensive, cumbersome and may not permit activities that a user may typically engage in, such as driving, for example.

Another such device is disclosed in U.S. Pat. No. 7,209,791 to Odom. The Odom '791 patent discloses an electromagnetic delivery system to influence a biological system. The system includes electrodes that transmit charges contained in a liquid container to be worn around the neck of a user to influence the body's electrical state. Similar to the device disclosed in the Rubins '445 patent, this system is a bulky apparatus that may likely have a minimal affect upon the body.

U.S. Pat. No. 7,131,744 to Lee and U.S. Pat. No. 7,001,056 to Clegg use batteries to illuminate jewels in darkened settings. U.S. Pat. No. 5,690,412 to Sheldon discloses added solar panels to keep batteries charged during the day while illuminating jewels at night. The jewelry disclosed in the Lee '744 patent, the Clegg '056 patent and the Sheldon '412 patent are for ornamental purposes only, and fail to disclose any health benefits.

Therefore, none of the references disclosed above have disclosed a portable and simple solution to influence brain wave states. Accordingly, there exists a need for portable electronic health devices that emit electro magnetic waves onto a wearer's body to optimize brain waves.

### SUMMARY OF THE INVENTION

In view of the foregoing background, it is therefore an object of the present invention to provide portable electronic health devices that emit electromagnetic waves onto a wearer's body to optimize brain waves. It is also an object of the present invention to provide a simple to use portable electronic health device that optimizes brain waves and that is not cumbersome. It is further an object of the present invention to provide a portable electronic health device that is affordable.

These and other objects, features, and advantages in accordance with the present invention are provided by a portable health device, comprising a housing and a power source connected to the housing. The portable health device may also include a frequency oscillation device in communication with the power source and an electromagnet carried by the housing and in communication with the power source.

The frequency oscillation device may be adapted to convert power from the power source to a pulse having a predetermined frequency. The pulse may charge the electromagnet to emit a pulsed electromagnetic wave onto a user.

The power source may include a plurality of photo voltaic cells or a battery, for example. The frequency oscillation device may comprise a printed circuit board, for example, and the electromagnet may include a ferrous core having at least one turn of wire.

In one embodiment, the pulse electromagnetic wave may have a frequency between about 7 Hz and 12 Hz. In another embodiment, the pulse electromagnetic wave may have a frequency between about 12 Hz and 36 Hz. In yet another embodiment, the pulse electromagnetic wave may have a frequency between about 4 Hz and 7 Hz. In still another embodiment, the pulse electromagnetic wave may have a frequency between about 1 Hz and 4 Hz. The pulse electromagnetic wave preferably has a frequency less than 50 Hz.

The housing preferably comprises a rigid material. More specifically, the rigid material may be metal or plastic. The housing and the power source of the device may be provided by jewelry to be worn by a user.

A method aspect of the present invention is for emitting a pulsed electromagnetic wave onto a user. The method comprises converting power from the power source connected to the housing to a pulse having a predetermined frequency using a frequency oscillation device. The method may also include using the pulse from the power source to charge the electromagnet to emit the pulse electromagnetic wave.

Accordingly, the portable health device and related methods of the present invention advantageously emit pulse electromagnetic waves having predetermined frequencies to optimize brain waves.

### BRIEF DESCRIPTION OF THE DRAWINGS

FIG. 1 is a front elevation view of the portable health device according to the present invention.

FIG. 1a is a side elevation view of the portable health device according to the present invention.

FIG. 2 is an electrical schematic of a portable health device according to the present invention.

FIG. 2a is a schematic of a printed circuit board of the portable health device according to the present invention.

FIG. 3 is a front perspective view of an electromagnet of the portable health device according to the present invention.

FIG. 3a is a schematic view of the electromagnetic shown in FIG. 3.

FIG. 4 is a portable health device according to the present invention taking the form of a bracelet to be worn by the user.

FIG. 5 is a front elevation view of another embodiment of the portable health device illustrated in FIG. 1.

FIG. 6 is a front elevation view of yet another embodiment of the portable health device illustrated in FIG. 1.

FIG. 7 is a front elevation view of still another portable health device according to the present invention.

### DETAILED DESCRIPTION OF THE PREFERRED EMBODIMENTS

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout, and prime and multiple prime notation is used to indicate similar elements in alternate embodiments.

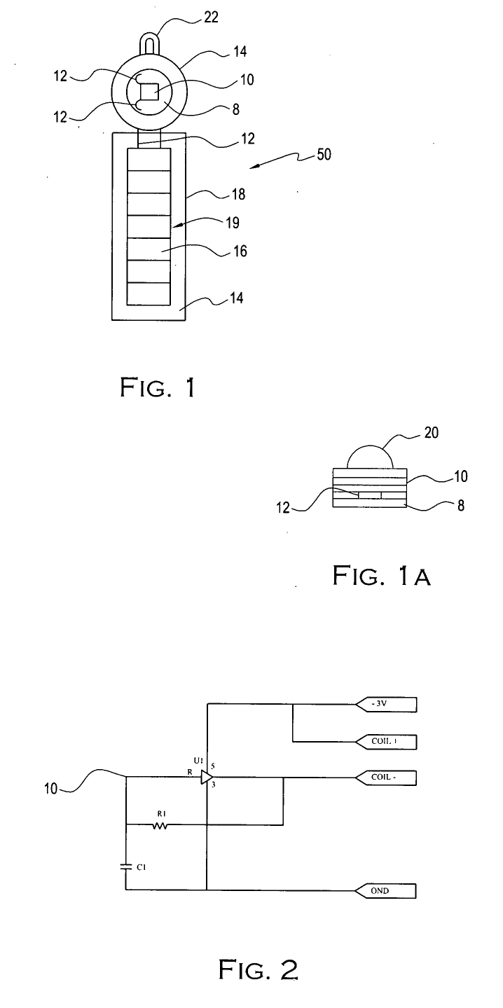

Referring initially to FIGS. 1 and 1a, a portable health device 50 is now described in great detail. The portable health device 50 according to the present invention illustratively includes a housing 18 and at least one power source 19 connected to the housing. The portable health device 50 may also include a frequency oscillation device 10 in communication with the power source 19. The portable health device 50 may further include an electromagnet 8 carried by the housing 18 and in communication with the power source 19. The frequency oscillation device 10 is preferably adapted to convert power from the power source 19 to a pulse having a predetermined frequency.

The pulse preferably charges the electromagnet 8 to emit a pulsed electromagnetic wave onto a user. The pulsed electromagnetic wave preferably has a predetermined frequency. As will be discussed in greater detail below, the pulsed electromagnetic wave may have any frequency, but is preferably substantially similar to the Alpha frequency of the user's brain waves. Accordingly, the present invention advantageously provides a portable electronic health device that emits electromagnetic waves onto the wearer's body to optimize brain waves.

As illustrated in FIG. 1, in one embodiment of the portable health device 50, the power source 19 may be provided by a plurality of photovoltaic cells, i.e., a solar power source. Further, the frequency oscillation device 10 may be provided by a printed circuit board. Those skilled in the art, however, will appreciate that the frequency oscillation device 10 may be provided by any other type of device capable of converting power from the power source to a pulse having a predetermined frequency.

As also illustrated in FIG. 1, the plurality of photovoltaic cells 16 are positioned in communication with the frequency oscillation device 10 using a plurality of wires 12. The frequency oscillation device 10 is also illustratively in communication with the electromagnet 8. Insulation 14 is preferably positioned adjacent the frequency oscillation device 10 and the electromagnet 8 to advantageously prevent a short.

As further illustrated in both FIG. 1 and FIG. 1a, the housing 18, the power source 19, the electromagnet 8, and the frequency oscillation device 10 are integrally formed as jewelry to be worn by the user. More specifically, the jewelry may include a jewel 20 positioned to overlie the frequency oscillation device 10, the electromagnet 8, and the wires 12 connecting the electromagnet to the frequency oscillation device.

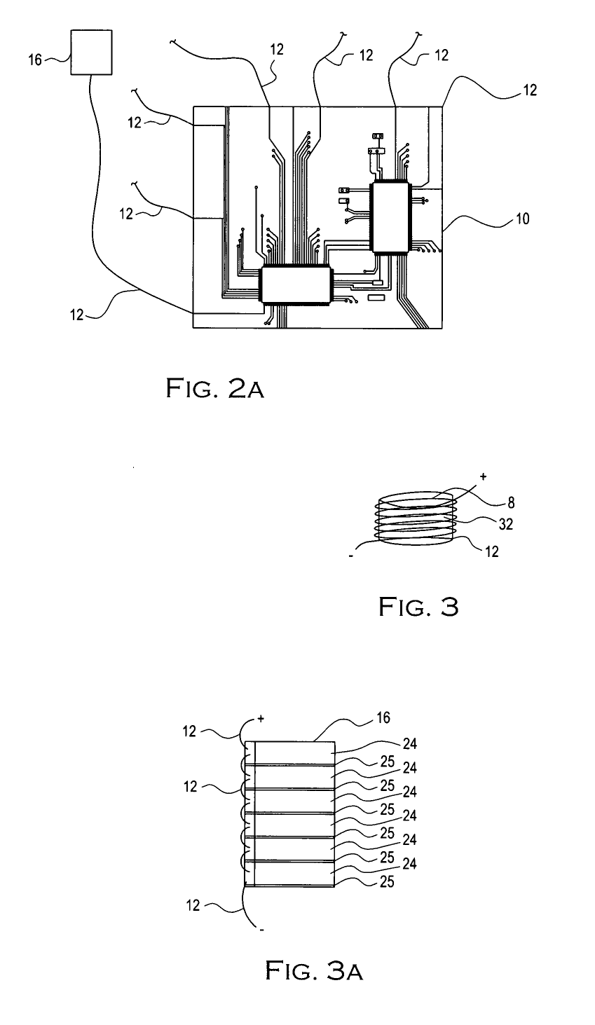

Referring now additionally to FIGS. 2-3a, additional features of the portable health device 50 are now described in greater detail. As illustrated in FIG. 2, the frequency oscillation device 10 is positioned in communication with the electromagnet 8. The electromagnet 8 may be provided by a ferrous core having at least one turn of wire, i.e., coil. Those skilled in the art, however, will appreciate that it is preferable to have a plurality of turns of wires surrounding the ferrous core. As illustrated in FIG. 2a, a plurality of wires 12 extend from the frequency oscillation device 10, e.g., the printed circuit board to make connections between the frequency oscillation device and the electromagnet 8.

More specifically, and as perhaps best illustrated in FIG. 2, the frequency oscillation device 10 uses capacitors and resistors needed to oscillate the preferred frequency within a desired predetermined range. FIG. 3 illustrates the electromagnet 8 with its ferrous core 32 and wire 12 wound around it to create an electromagnet. FIG. 3a illustrates the solar panel 16 made up of solar cells 24 with gaps between each cell 25. The solar cells 24 may be soldered together in a series with wire 12 to create output power when light strikes the solar array. It also shows the two wires 12 terminating in positive (+) and negative (−) leads.

The housing 18 is preferably provided by rigid material. More specifically, the rigid material is preferably either metal or plastic. Those skilled in the art, however, will appreciate that the rigid material may be provided by any other type of material.

The jewelry may take the form of a necklace, a bracelet, or any other form of jewelry as understood by those skilled in the art. Further, the jewelry is preferably adapted to be worn by humans, but those skilled in the art will appreciate that any biological life form may wear the jewelry as well and still obtain the same benefits. For example, the jewelry may be used for pets or any other type of biological life form as understood by those skilled in the art.

In the preferred embodiment, the jewelry is provided as a necklace to be worn by the user. When the portable health device 50 is worn as jewelry, a top hook 22 may extend upwardly from a top portion of the portable health device for connection to a chain, for example, or any other string material to be worn by the user.

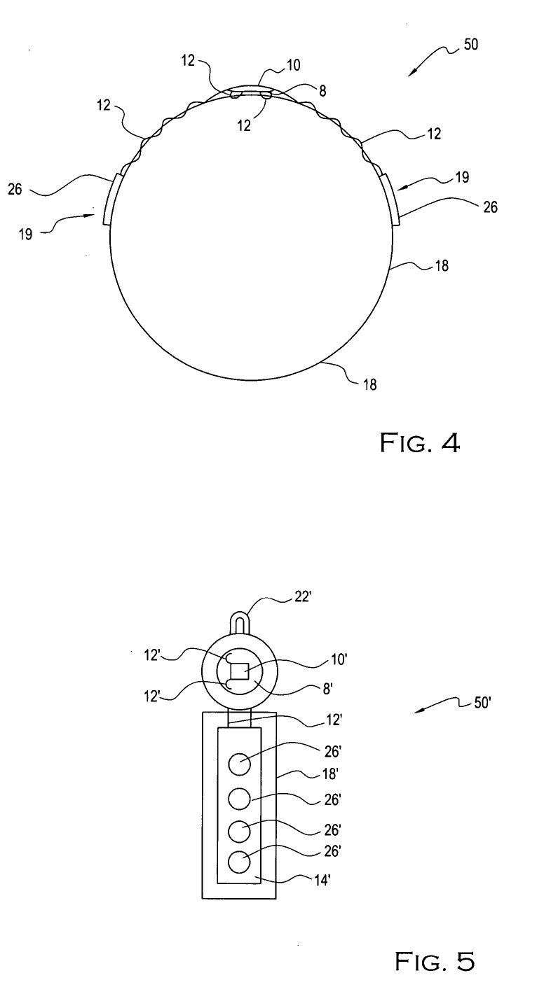

In other embodiments, the portable health device 50 may take the form of a bracelet, as illustrated, for example, in FIG. 4. When the portable health device 50 takes the form of a bracelet, the power source 19 may be provided by batteries 26 carried by a body portion of the bracelet. A plurality of wires 12 are used to provide connections between the batteries 26, the electromagnet 8, and the frequency oscillation device 10. Those skilled in the art will appreciate that the portable health device 50 of the present invention may take the form of any type of jewelry as well.

The pulse electromagnetic wave emitted from the electromagnet 8 may have many varying frequencies. For example, one embodiment of the invention emits a pulse electromagnetic wave having a frequency between 7 Hz and 12 Hz. Another embodiment of the invention emits a pulse electromagnetic wave having a frequency between about 12 Hz and 36 Hz. Yet another embodiment of the invention emits a pulse electromagnetic wave having a frequency between about 4 Hz and 7 Hz, and still another embodiment of the invention emits a pulse electromagnetic wave having a frequency between about 1 Hz and 4 Hz. It is preferable, however, that the pulse electromagnetic wave has a frequency less than about 50 Hz. Those skilled in the art, however, will appreciate that the pulse electromagnetic wave emitted by the electromagnet 8 may have any frequency.

The frequencies listed above, however, are preferable as they enhance stimulation of brain waves. These frequencies generally relate to the frequency range for Alpha waves, Beta waves, Delta waves, and Theta waves. The present invention, however, advantageously contemplates the emission of pulses having frequencies outside of these ranges.

Referring now additionally to FIG. 5, another embodiment of the portable health device 50′ is now described in greater detail. The embodiment of the portable health device 50′ illustrated in FIG. 5 shows the power source 19′ being provided by a plurality of batteries 26′. The batteries 26′ are preferably surrounded by insulating material 14′ to prevent possible shorting. Further, the batteries 26′ may be connected to the frequency oscillation device 10′ using wires 12′. The other elements of this embodiment of the portable health device 50′ are similar to those elements of the first embodiment of the portable health device 50, are labelled using prime notation and require no further discussion herein.

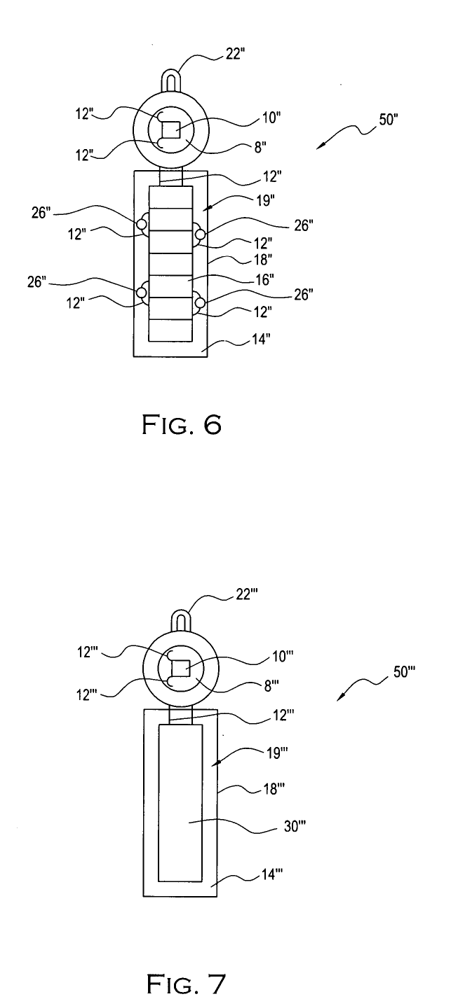

Referring now additionally to FIG. 6, yet another embodiment of the portable health device 50″ is now described in greater detail. The embodiment of the portable health device 50″ illustrated in FIG. 6 shows the power source 19″ being provided by a combination of a solar panel 16″ having a plurality of photovoltaic cells and a plurality of batteries 26″. In this particular embodiment, the solar panels 16″ may be used to charge the batteries 26″, or may be used to provide power to the portable health device 50″. When the batteries 26″ are charged by the solar panel 16″, the power necessary to run the frequency oscillation device 10″ may be provided from the batteries.

The solar panel 16″ and the batteries 26″ are preferably surrounded by insulating material 14″ to prevent possible shorting. Further, the solar panel 16″ and the batteries 26″ may be connected to the frequency oscillation device 10″ using wires 12″. The other elements of this embodiment of the portable health device 50″ are similar to those elements of the first embodiment of the portable health device 50, are labelled using double prime notation and require no further discussion herein.

Referring now additionally to FIG. 7, still another embodiment of the portable health device 50′″ is now described in greater detail. The embodiment of the portable health device 50′″ illustrated in FIG. 7 shows the power source 19′″ being provided by a fuel cell 30′″. In this particular embodiment, the fuel cell 30′″ may be connected to the frequency oscillation device 10′″ using wires 12′″. The other elements of this embodiment of the portable health device 50′″ are similar to those elements of the first embodiment of the portable health device 50, are labelled using triple prime notation and require no further discussion herein.

A method aspect of the present invention is for emitting a pulsed electromagnetic wave onto a user. The method may include converting power from a power source 19 connected to a housing 18 to a pulse having a predetermined frequency using a frequency oscillation device 10. The method may also include using the pulse from the power source 19 to charge at least one electromagnet 8 to emit the pulsed electromagnetic wave.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. A portable health device comprising:
a housing;
at least one power source connected to said housing;
a frequency oscillation device in communication with said power source; and
at least one electromagnet carried by said housing and in communication with said at least one power source;
wherein said frequency oscillation device is adapted to convert power from the power source to a pulse having a predetermined frequency; and
wherein the pulse charges said at least one electromagnet to emit a pulsed electromagnetic wave onto a user.

2. A device according to claim 1 wherein said at least one power source comprises at least one of a plurality of photovoltaic cells, at least one battery and at least one fuel cell.

3. A device according to claim 1 wherein said frequency oscillation device comprises at least one printed circuit board.

4. A device according to claim 1 wherein said at least one electromagnet includes a ferrous core having at least one turn of wire.

5. A device according to claim 1 wherein said housing comprises rigid material.

6. A device according to claim 5 wherein the rigid material is at least one of metal and plastic.

7. A device according to claim 1 wherein the pulsed electromagnetic wave has a frequency between about 7 Hz and 12 Hz.

8. A device according to claim 1 wherein the pulsed electromagnetic wave has a frequency between about 12 Hz and 36 Hz.

9. A device according to claim 1 wherein the pulsed electromagnetic wave has a frequency between about 4 Hz and 7 Hz.

10. A device according to claim 1 wherein the pulsed electromagnetic wave has a frequency between about 1 Hz and 4 Hz.

11. A device according to claim 1 wherein the pulsed electromagnetic wave has a frequency less than 50 Hz.

12. A device according to claim 1 wherein said housing, said at least one power source, said at least one electromagnet, and said frequency oscillation device are integrally formed as jewelry to be worn by the user.

13. A method of emitting a pulsed electromagnetic wave onto a user, the method comprising:
converting power from a power source connected to a housing to a pulse having a predetermined frequency using a frequency oscillation device; and
using the pulse from the power source to charge at least one electromagnet to emit the pulsed electromagnetic wave.

14. A method according to claim 13 wherein the at least one power source comprises at least one of a plurality of photovoltaic cells, at least one battery, and at least one fuel cell.

15. A method according to claim 13 wherein the frequency oscillation device comprises at least one printed circuit board; and wherein the at least one electromagnet includes a ferrous core having at least one turn of wire.

16. A method according to claim 13 wherein the pulsed electromagnetic wave has a frequency between about 7 Hz and 12 Hz.

17. A method according to claim 13 wherein the pulsed electromagnetic wave has a frequency between about 12 Hz and 36 Hz.

18. A method according to claim 13 wherein the pulsed electromagnetic wave has a frequency between about 4 Hz and 7 Hz.

19. A method according to claim 13 wherein the pulsed electromagnetic wave has a frequency between about 1 Hz and 4 Hz.

20. A method according to claim 13 wherein the pulsed electromagnetic wave has a frequency less than 50 Hz.
